(19) United States
(12) Patent Application Publication
(10) Pub. No.: US 2014/0261348 A1
(43) Pub. Date: Sep. 18, 2014

(54) Exhaust Gas Sensor Device

(71) Applicant: Robert Bosch GmbH, Stuttgart (DE)

(72) Inventors: Thomas Wahl, Pforzheim (DE); Richard Fix, Gerlingen (DE); Denis Kunz, Untergruppenbach (DE); Alexander Martin, Regensburg (DE)

(73) Assignee: Robert Bosch GmbH, Stuttgart (DE)

(21) Appl. No.: 14/200,166

(22) Filed: Mar. 7, 2014

(30) Foreign Application Priority Data
Mar. 14, 2013 (DE) 10 2013 204 480.1

(51) Int. Cl. F02D 35/00; G01N 33/00

(52) U.S. Cl. CPC F02D 35/0015; G01N 33/0009
USPC 123/672; 73/23.31

(57) ABSTRACT

An exhaust gas sensor device for recording a concentration of at least one exhaust gas component in an exhaust system of an internal combustion engine includes at least one exhaust gas sensor with intrinsic signal amplification. The at least one exhaust gas sensor records the concentration of at least one exhaust gas component.

This application claims priority under 35 U.S.C. §119 to patent application no. DE 10 2013 204 480.1, filed on Mar. 14, 2013 in Germany, the disclosure of which is incorporated herein by reference in its entirety.

The present disclosure relates to an exhaust gas sensor device for recording a concentration of at least one exhaust gas component in the exhaust system of an internal combustion engine and to an exhaust system of an internal combustion engine.

### BACKGROUND

Exhaust gas sensors that are used in modern or future exhaust aftertreatment systems must be able to detect harmful gases such as nitrogen oxides with high accuracy of a few ppm. Current ceramic-based sensor concepts use ionic pump currents as a sensor signal, which change depending on the concentration of nitrogen oxides in the exhaust gas. In principle, such sensor elements only provide very small sensor signals (currents in the nA range), so that signal amplification or signal processing close to the sensor in an additional sensor control unit is essential. The measured sensor signals can thus only be passed to the central engine controller after suitable preparation. Said additional electronic unit is consequently connected with high development or manufacturing costs.

### SUMMARY

The subject matter of the present disclosure is an exhaust gas sensor device for recording a concentration of at least one exhaust gas component in the exhaust system of an internal combustion engine, which comprises at least one exhaust gas sensor with intrinsic signal amplification, wherein at least one exhaust gas sensor records the concentration of at least one exhaust gas component.

The exhaust gas sensor can e.g. record an analyte gas species of an exhaust gas and can thus record the concentration of ionized, i.e. charged, gas species on a sensor surface depending on the partial pressure of the analyte gas in the surroundings. The presence of an analyte gas can thus cause a change in potential, which can be recorded by the exhaust gas sensor. Intrinsic signal amplification means that said change in potential, which occurs as a result of the charge generated by the analyte gas on the gate electrode, can in turn be directly converted into a change in channel current, wherein at the same time signal amplification can take place. Consequently, no direct current measurement takes place, as is the case e.g. with ceramic exhaust gas sensors. Using the intrinsic signal amplification, sensor currents in the micro- to milliampere range and sensor signals can thus be output by means of changes in current in the sub-milliampere or microampere range. In this way the currents or the changes in current can be directly read into the engine controller. Thus additional amplification, e.g. by means of a sensor control unit, can be eliminated when using an exhaust gas sensor as described above. Furthermore, the susceptibility to faults of the exhaust system can be reduced because there is no sensor control unit.

Advantageously, the at least one exhaust gas sensor is a ChemFET sensor. The term ChemFET sensor denotes a chemically sensitive field effect transistor (in short ChemFET). The ChemFET sensor is a special form of a field effect transistor, which is used as a sensor for chemicals and chemical properties of substances. The ChemFET sensors use chemically active semiconducting gas sensors based on field effect transistors, with which gas molecules interact with a catalytically active gate electrode. Said absorptions thereby cause a change of the effectively applied gate potential, which in turn can be measured in a change of the source-drain current (sensor signal). Thereby the change in potential is intrinsically amplified by the field effect, so that a current change in the microampere to milliampere range results, depending on the design of the field effect transistor and depending on the gases to be absorbed. ChemFET sensors can thus detect gas components in very small concentrations (single digit ppm range).

The ChemFET sensor can be based on a field effect transistor, whose gate electrode selectively interacts with the gases to be detected. The catalytic activity of the gate electrode can e.g. cause dissociation of the analyte gas species and can thus change the concentration of ionized, i.e. charged gas species on the sensor surface depending on the partial pressure of the analyte gas in the surroundings. The presence of an analyte gas can thus lead to a change in potential on the gate of the field effect transistor. Said change in potential can in turn be directly converted into a change in channel current, wherein at the same time signal amplification can take place. The gain can thereby be dependent on the transfer characteristic of the field effect transistor being used.

Using said intrinsic signal amplification, sensor currents in the microampere to milliampere range and sensor signals can be output by means of changes in current in the sub-milliampere or microampere range. This enables the currents or the changes in current to be directly read into the engine controller. Thus additional amplification, e.g. by a sensor control unit, can be eliminated when using a ChemFET sensor. Furthermore, the susceptibility to faults of the exhaust system can be reduced because there is no sensor control unit.

In an advantageous embodiment of the exhaust gas sensor device, the at least one exhaust gas sensor is integrated on at least one sensor chip. In this way the size of the exhaust gas sensor device can be reduced and simplified, whereby installation space can be saved in the exhaust system. The use of robust semiconducting materials with a wide band gap, e.g. selected from a group containing silicon carbide (SiC) or gallium nitride (GaN), can enable the use of an exhaust gas sensor even at high temperatures and thus for measuring hot gases in corrosive environments, such as combustion exhaust gas.

Advantageously, the exhaust gas sensor device is not connected to a sensor control unit. The at least one exhaust gas sensor can be designed such that intrinsic signal amplification of the exhaust gas sensor through the use of an additional electronic circuit close to the sensor, especially for amplification of the sensor signal, can be eliminated. The great advantage consequently lies in saving the development and manufacturing costs of the sensor control unit close to the sensor. Moreover, additional installation space in the exhaust system that would be required to install the sensor control unit close to the sensor can be saved.

It is advantageous if a plurality of different exhaust gas sensors can be integrated on the at least one sensor chip of the exhaust gas sensor device. In this way different exhaust gas components can be selectively measured. Thereby the exhaust gas sensors can measure e.g. nitrogen oxides, hydrocarbons, carbon monoxide and/or ammonia.

Thus an exhaust gas sensor device that can record a plurality of exhaust gas components can be implemented in a simple manner.

Advantageously, the at least one sensor chip of the exhaust gas sensor device comprises a circuit for the signal processing of recorded sensor values. In this way the signal processing of the recorded sensor signals can take place directly on the at least one sensor chip. Thus a sensor control unit can be eliminated and the sensor signals processed by means of the at least one sensor chip can be directly forwarded to the engine controller.

In another advantageous embodiment of the exhaust gas sensor device, the different exhaust gas sensors on the at least one sensor chip can be read out one after the other by means of a multiplexer. The term multiplexer denotes a selection circuit with which one input signal is selected from a number of input signals and is switched through to the output. Multiplexers are comparable with rotary switches that are not operated by hand but by electronic signals. The difference from a relay is that the connections are not mechanical but implemented by integrated semiconducting circuits. By the use of a multiplexer, the at least one sensor chip can read out the sensor signals of the exhaust gas sensors in succession, can process them and pass them to the engine control unit by means of an output signal. In this way a sensor control unit for processing and forwarding the sensor signals of the exhaust gas sensor device can be eliminated. Because of the wide band gap of the semiconducting material, the function of the multiplexer can be implemented directly on the sensor chip by means of electronics suitable for high temperatures, e.g. semiconducting elements, diodes or transistors. Furthermore, the number of cables for the operation of a sensor chip with different exhaust gas sensors can be reduced.

It is advantageous if the at least one sensor chip of the exhaust gas sensor device comprises yet more functions besides the recording of concentrations of at least one exhaust gas component. In the same way, other functions can be implemented that are processed directly on the sensor chip, e.g. analog to digital converters, units for communication with the bus system used, sensor temperature regulators, signal filtering and modulation. Thus all the signal processing can be integrated on the at least one sensor chip, whereby a sensor control unit close to the sensor for signal processing of the sensor signals of the exhaust gas sensor device can be eliminated. Furthermore, because of the wide band gap of the semiconducting material the other functions can be implemented directly on the sensor chip by means of electronics suitable for high temperatures.

Advantageously, part of the signal processing of recorded sensor values of the at least one sensor chip of the exhaust gas sensor device can be moved into a central engine controller. Because of the intrinsic amplification and thus the good signal-to-noise ratio, part of the signal processing can be moved into the central engine controller. For this purpose, an analog data stream can be output by the sensor, which is further processed in the engine controller. Alternatively, the digitization of the sensor signals can be implemented directly on the sensor chip and then a digital data stream of the raw sensor signal can be output via e.g. a SENT interface or a PSIS interface directly into the central engine controller. The ChemFET sensor can thus output signals that can be directly interpreted and further processed by the central engine controller, so that a controller dedicated to the sensor is eliminated.

In another advantageous embodiment of the exhaust gas sensor device, the signal processing can be integrated on a second sensor chip. In this way, the exhaust gas sensor device can be simplified.

Furthermore, the disclosure comprises an exhaust system of an internal combustion engine with at least one engine controller, wherein the engine controller controls an engine; an exhaust pipe, wherein exhaust gases of the engine are passed through the exhaust pipe; an exhaust gas sensor device, wherein the exhaust gas sensor device determines a concentration of at least one exhaust gas component in the exhaust pipe, wherein the exhaust gas sensor device is directly connected to the engine controller. The exhaust gas sensor device can thus deliver sensor signals which can be processed directly in the central engine controller. Thus a sensor control unit for signal amplification and/or signal processing for forwarding the sensor signals from the exhaust gas sensor device to the engine controller can be eliminated, whereby the design of the exhaust system is simplified. Furthermore, the susceptibility to faults of the exhaust system can be reduced because there is no sensor control unit.

In an advantageous embodiment of the exhaust system, the exhaust system comprises no sensor control unit. By eliminating a sensor control unit, additional installation space in the exhaust system can be eliminated, because the exhaust gas sensor device can forward the sensor signals directly without an intermediately connected sensor control unit. In this way, the structure of the exhaust system can be simplified and also reduced. Furthermore, the susceptibility to faults of the exhaust system can be reduced because there is no sensor control unit.

Advantageously, the exhaust gas sensor device of the exhaust system comprises at least one ChemFET sensor. Thus additional amplification, e.g. by a sensor control unit, can be eliminated when using at least one ChemFET sensor. Furthermore, the susceptibility to faults of the exhaust system can be reduced because there is no sensor control unit.

Regarding other features and advantages of the subject matter according to the disclosure, reference is hereby explicitly made to the explanations in connection with the exhaust gas sensor device according to the disclosure and the exhaust system according to the disclosure as well as to the examples.

### BRIEF DESCRIPTION OF THE DRAWINGS

Further advantages and advantageous embodiments of the subject matters according to the disclosure are illustrated by the figures and the examples and explained in the following description. It should be noted thereby that the figures and the examples have only a descriptive character and are not intended to restrict the disclosure in any way. In the figures

FIG. 1 shows a schematic view of a section of an exhaust system of the prior art;

FIG. 2 shows a schematic view of a section of an exhaust system according to the present disclosure; and

FIG. 3 shows a schematic view of a sensor chip according to the present disclosure.

### DETAILED DESCRIPTION

In FIG. 1 a schematic view of a section of an exhaust system 10 of an internal combustion engine of the prior art is illustrated. The exhaust system 10 comprises thereby an engine controller 12 for controlling an engine and a sensor control unit 20 connected to the engine controller 12. The sensor control unit 20 is furthermore connected to a sensor element 22, which detects an exhaust gas in an exhaust pipe 14. The exhaust gas is illustrated in FIG. 1 as an arrow within the exhaust pipe 14. In the prior art a ceramic-based sensor element 22 is used, which uses ionic pump currents as a sensor signal. For this reason, such sensor elements 22 only provide small sensor signals in the nA range. The sensor signals are forwarded to the sensor control unit 20. In the sensor control unit 20, the sensor signals are amplified and/or processed before the sensor signals are forwarded to the engine controller 12. Using the sensor signals e.g. the motor of the internal combustion engine can be controlled.

In FIG. 2 a schematic view of a section of an exhaust system of an internal combustion engine according to the present disclosure is illustrated. The exhaust system 10 comprises at least one engine controller 12, wherein the engine controller controls an engine, an exhaust pipe 14, wherein exhaust gases of the engine are passed through the exhaust pipe 14, and an exhaust gas sensor device 30, wherein the exhaust gas sensor device determines a concentration of at least one exhaust gas component in the exhaust pipe 14. The exhaust gas is illustrated in FIG. 2 as an arrow within the exhaust pipe 14.

In contrast to FIG. 1, it can be seen in FIG. 2 that the exhaust system, instead of comprising an exhaust gas sensor device 22 connected to a sensor control unit 20, comprises only an exhaust gas sensor device 30 that is directly connected to the engine controller 12. The exhaust gas sensor device 30 thereby comprises at least one exhaust gas sensor with intrinsic signal amplification, wherein the at least one exhaust gas sensor records the concentration of at least one exhaust gas component. The exhaust gas sensor is at least one ChemFET sensor in this exemplary embodiment. The exhaust system 10 comprises no sensor control unit 20 as in the exhaust system of the prior art in FIG. 1, whereby the susceptibility to faults of the exhaust system 10 is reduced because there is no sensor control unit 20. Furthermore, the installation space of the exhaust system 10 can also be reduced.

A sensor chip 32 of the exhaust gas sensor of the exhaust gas sensor device 30 is illustrated schematically in FIG. 3. The exhaust gas sensor device 30 in FIG. 3 comprises a sensor chip 32, wherein a plurality of different ChemFET sensors 36a, 36b, 36c, 36d are integrated on the sensor chip 32. The ChemFET sensors can thereby identify different exhaust gas components in the exhaust gas to be detected. For example, ChemFET sensor 36a can detect nitrogen oxides, ChemFET sensor 36b can detect hydrocarbons, ChemFET sensor 36c can detect carbon monoxide and ChemFET sensor 36d can detect ammonia. The ChemFET sensors consist of robust semiconducting materials with a wide band gap, e.g. selected from a group containing SiC or GaN, whereby the ChemFET sensors can also be used at higher temperatures and hence for measuring hot gases in corrosive environments such as combustion exhaust gas.

Furthermore, the sensor chip 32 of the exhaust gas sensor device 30 comprises at least one circuit for signal processing in order to record sensor signals. In this way, the signal processing of the recorded sensor signals can take place directly on the sensor chip 32. FIG. 3 shows that the different ChemFET sensors 36a, 36b, 36c, 36d on the sensor chip 32 of the exhaust gas sensor device 30 can be read out one after the other by means of a multiplexer 34. The multiplexer 34 provides an output signal to the engine controller 12, whereby the engine controller controls the engine accordingly. By using a multiplexer 34, the number of cables required for the operation of a sensor chip 32 with a plurality of ChemFET sensors 36a, 36b, 36c, 36d can be reduced. Because of the wide band gap of the semiconducting material, the function of the multiplexer 34 can be implemented directly on the sensor chip 32 by means of electronics suitable for high temperatures, e.g. transistors or diodes.

In a not illustrated exemplary embodiment the sensor chip 32 of the exhaust gas sensor device 30 comprises yet more functions besides recording concentrations of at least one exhaust gas component. The other functions are implemented on the sensor chip 32 by means of electronics suitable for high temperatures and can comprise e.g. analog to digital converters, units for communications with different bus systems as well as sensor temperature controllers. Hence the entire signal processing is integrated on the sensor chip 32.

In another not illustrated exemplary embodiment, a part of the signal processing of recorded sensor values of the sensor chip 32 of the exhaust gas sensor device 30 is moved into the central engine controller 12. Because of the intrinsic amplification and the resulting good signal-to-noise ratio of the sensor signals of the ChemFET sensors, these can be directly interpreted and further processed by the central engine controller. An analog or digital data stream is thereby forwarded directly into the central engine controller 12 or a digital data stream can be passed via a SENT interface or a PSIS interface directly into the central engine controller 12. This eliminates the use of a sensor control unit 20 for the signal amplification and/or signal processing of the sensor signals of the exhaust gas sensor device 30 for forwarding to the central engine controller 12.

In another not illustrated exemplary embodiment, the signal processing can be integrated on a second sensor chip of the exhaust gas sensor device 30. The simple circuits for signal processing can thereby be integrated within a second sensor chip directly in the plug. In this way, no additional installation space for a sensor control unit 20 in the exhaust system 10 is required, whereby more compact exhaust systems can be constructed. Furthermore, the exhaust system 10 is less susceptible to faults because there is no sensor control unit 20.

What is claimed is:

1. An exhaust gas sensor device for recording concentrations of exhaust gas components in an exhaust system of an internal combustion engine, comprising:
at least one exhaust gas sensor with intrinsic signal amplification, and configured to record a concentration of at least one exhaust gas component.

2. The exhaust gas sensor device according to claim 1, wherein the at least one gas sensor is a chemically sensitive field effect transistor sensor.

3. The exhaust gas sensor device according to claim 1, wherein the at least one exhaust gas sensor is integrated on at least one sensor chip.

4. The exhaust gas sensor device according to claim 1, wherein the exhaust gas sensor device fails to be connected to a sensor control unit.

5. The exhaust gas sensor device according to claim 3, wherein a plurality of different exhaust gas sensors is integrated on the at least one sensor chip.

6. The exhaust gas sensor device according to claim 3, wherein the at least one sensor chip comprises a circuit configured to perform at least part of a signal processing of recorded sensor values.

7. The exhaust gas sensor device according to claim 5, wherein the plurality of different exhaust gas sensors integrated on the at least one sensor chip is configured to be read out sequentially using a multiplexer.

8. The exhaust gas sensor device according to claim 3, wherein the at least one sensor chip comprises at least one circuit that fails to be configured for recording of concentrations of at least one exhaust gas component.

9. The exhaust gas sensor device according to claim 6, wherein a central engine controller is configured to perform at least part of the signal processing.

10. The exhaust gas sensor device according to claim 1, wherein a second sensor chip comprises a circuit configured to perform at least part of a signal processing of recorded sensor values.

11. An exhaust gas system of an internal combustion engine, comprising:
an engine controller configured to control an engine;
an exhaust pipe configured to pass exhaust gasses of the engine therethrough;
an exhaust gas sensor device directly connected to the engine controller, and configured to determine a concentration of at least one exhaust gas component in the exhaust pipe.

12. The exhaust gas system according to claim 11, wherein the exhaust system fails to comprise a sensor control unit.

13. The exhaust gas system according to claim 11, wherein the exhaust gas sensor device includes at least one chemically sensitive field effect transistor sensor.
